(19) United States
(12) Patent Application Publication
(10) Pub. No.: US 2020/0321087 A1
(43) Pub. Date: Oct. 8, 2020

(54) SYSTEM AND METHOD FOR RECURSIVE MEDICAL HEALTH DOCUMENT RETRIEVAL AND NETWORK EXPANSION

(71) Applicant: MOXE HEALTH CORPORATION, Madison, WI (US)

(72) Inventors: Tomas C. WILLIS, Madison, WI (US); Daniel P. WILSON, Madison, WI (US)

(21) Appl. No.: 16/838,126

(22) Filed: Apr. 2, 2020

Related U.S. Application Data
(60) Provisional application No. 62/828,829, filed on Apr. 3, 2019.

(51) Int. Cl. G16H 10/60; G16H 70/00; G16H 15/00; G16H 40/63; G16H 40/67; G06F 40/205; G06F 16/38

(52) U.S. Cl. CPC G16H 10/60; G16H 70/00; G16H 15/00; G06F 16/38; G16H 40/67; G06F 40/205; G16H 40/63

(57) ABSTRACT

The present disclosure is directed to systems and methods for medical health document retrieval and network expansion. In a method, a computing system receives a request for a health record, obtains contextual information associated with the request, determines a source of health record information for the health record based on the request and contextual information, retrieves the health record information from the source, and assembles the health record using the retrieved health record information. In some embodiments, the method may be recursive and analyze the retrieved health record information for references to additional health record information that may not be present in the health record.

### CROSS-REFERENCE TO RELATED PATENT APPLICATIONS

The present application claims the benefit of U.S. Provisional Application No. 62/828,829, filed on Apr. 3, 2019, entitled “System and Method for Recursive Medical Health Document Retrieval and Network Expansion,” the contents of which are incorporated by reference herein in their entirety.

### FIELD OF THE DISCLOSURE

The present disclosure relates generally to electronic record keeping. More particularly, the present disclosure related to systems and methods of retrieving medical documents.

### BACKGROUND

Health records can be contained in many forms in a variety of databases each being maintained by separate health care organizations (HCO). For example, a hospital may maintain medical records or documents regarding a first diagnosis for a first patient and a lab may maintain records or documents regarding test results for the first patient. However, the health record maintained by the hospital may be incomplete because it does not include the lab results. Similarly, the health record for the first patient maintained by the lab may be incomplete because it does not include medical records regarding the first diagnosis. Thus, the first patient and medical providers may not have ready access to an aggregate health record stored or maintained by an HCO.

### BRIEF DESCRIPTION OF THE DRAWINGS

Like reference numbers and designations in the various drawings indicate like elements. For purposes of clarity, not every component is necessarily labeled in every drawing. In the drawings:

FIG. 1 depicts a health records network in accordance with an illustrative embodiment of the present disclosure.

FIG. 2 depicts a health record in accordance with an illustrative embodiment of the present disclosure.

FIG. 3 depicts additional details of a health records system including a health document expansion retrieval system in accordance with an illustrative embodiment of the present disclosure.

FIG. 4 depicts a flow diagram of a method of operating the health document expansion retrieval system in accordance with an illustrative embodiment of the present disclosure.

FIG. 5 depicts an example flow of operations of the health document expansion retrieval system in accordance with an illustrative embodiment of the present disclosure.

FIG. 6 depicts an additional workflow of operation 133 of FIG. 5 in accordance with an illustrative embodiment of the present disclosure.

FIG. 7 depicts an additional workflow of operation 143 of FIG. 5 in accordance with an illustrative embodiment of the present disclosure.

FIG. 8 depicts an additional workflow of operation 153 of FIG. 5 in accordance with an illustrative embodiment of the present disclosure.

FIG. 9 is a block diagram illustrating physical components (e.g., hardware) of a computing device in accordance an illustrative embodiment of the present disclosure.

### DETAILED DESCRIPTION

The ensuing description provides embodiments only and is not intended to limit the scope, applicability, or configuration of the claims. Rather, the ensuing description will provide those skilled in the art with an enabling description for implementing the embodiments. It being understood that various changes may be made in the function and arrangement of elements without departing from the spirit and scope of the appended claims.

In health records systems, Health Care Organizations (“HCO”) (health plans, health systems, provider(s), employers, medical labs, pharmacies, nursing homes, long-term care facilities, digital health companies, individual patients and families, pharmaceutical and medical test labs, genomics services, university researchers, pharmaceutical researchers and manufacturers, medical device manufacturers, etc.) maintain separate records systems for the individuals (also referred to as patients, members, subjects, etc.) they have responsibilities for or otherwise provide care for. An individual may be identified by separate identifiers in each system. When data needs to be exchanged between systems, for example when a health plan needs to confirm a diagnosis associated with an insurance claim by asking a health system for confirming evidence, it may not be clear which member entity in the payor records system corresponds to which patient entity in the health system database. A health records network spanning from the records systems of various health care organizations may connect the data sources to provide a more complete record of a patient's clinical history. Expanding the health records network allows more complete clinical histories to be gathered for more patients, whether on behalf of the patients or another organization or entity.

In accordance with examples of the present disclosure, a system and method for retrieving clinical documents from healthcare organizations, identifying references to further documentation, identifying the location of the further documentation, gaining access to new locations of documentation, and gathering the further documentation to provide the most complete record of clinical data is provided. A requesting entity, or requester, such as a health insurance system, may need to request a medical document for an individual from a custodian of health records. The document requested may require multiple health records for different events and may contain various types of health records, containing different types of data (text, images, audio recordings, video recordings, genomics data, spreadsheets, etc.) in discrete, structured or unstructured formats, across a range of known types of health and personal data, including, but not limited to, clinical records, demographic data, claims data, other billing data, pharmacy data, genomic data, dental data, social determinants. Health records may include any documents, materials, or information related to health care billing information, health care treatment information, health care information regarding prior conditions, or any other information related to the health or care that one or more patients have or will have received. The custodian of the health care document sought by the requester may be a medical provider or health system, such as a hospital or medical practice, or a patient, or entity acting on behalf of a patient. The custodian may be a shared health data system like a Health Information Exchange (HIE), a lab, digital health company, pharmacy, governmental agency or other source of health record documents.

Additionally, a health system may request medical documents held by a health plan or other steward of health records, or a pharmaceutical company may make a request for data stored in the EMR of a nursing home where a clinical trial participant is receiving care. More broadly, any health care organization or patient or entity working on behalf of a patient or acting under subpoena in a legal proceeding may make a request for documents from a health care organization.

Each medical document corresponds to a document reference. Each document reference includes a description of the document and a location, or source, on the health records network. Such documents may be retrieved digitally from the data source, from an electronic medical records system (EMR) or laboratory information system (LIS), or manually, in which case such records may be converted into an electronic record by a document digitizer. These health records can be retrieved via a streaming data source in the form of messages, as with an HL7 v2 stream, or as a standardized clinical record like a CDR or C-CDA, by standardized interfaces like IHE profiles and FHIR or by proprietary means defined by EMR vendors, health plans and other stewards or custodians of health records.

Each data source may correspond to a node in a health records network and each health records custodian may correspond to one or more source nodes in the health records network. Each requesting entity may correspond to one or more target nodes in a health records network. Thus, the health records network includes nodes that take roles of sources (places where documents are) or targets (requesters of data). Each node has associated access settings and credentials that are required for the system to access the node. In some cases, a given node may operate as a source under some circumstances and as a target under other circumstances.

Each requester may or may not have rights to a document they are requesting from the data source. It is important to determine the eligibility of a requester to access a given health records document or some or all of the parts of the document. Eligibility is an outcome of business and legal agreements and rules in place that apply to the requester and the custodian of the data source. Additional eligibility rules may be specified by other parties in the health records network, including the individual (patient, member, subject, etc.) and/or their legal guardian or legal representative. The eligibility rules between the requester and the health records custodians may be specified in the health records network as a set of rules and configuration data. Likewise, eligibility rules specified by the individual/legal guardian/representative may be specified in the health records network as a set of rules and configuration data. At the time of an initial request for a document, the eligibility rules that apply to a given document request may already be known to the health records network.

Each document treated by the health document expansion retrieval system is examined to determine if it contains references to further documentation and/or or other health records, other documents, and/or or other sources of information. That is, a document includes health record information and the health record information may be examined to determine if it contains references to additional relevant health record information that is absent in the document. In some embodiments, the additional health record information or document(s) containing the additional health record information is indicated by a document reference. In some embodiments, the document reference includes a description of the additional health record information or document(s) containing the additional health record information and a location of the additional health record information or document(s) containing the additional health record information. In some embodiments, the identification of the location may be determined based on the contextual information of the examined document. In some embodiments, the location is determined based on the contextual information of the document reference. In some embodiments, the location may include a source that indicates an electronic address on the network of a health record provider (i.e. custodian) of the document. In still further embodiments, the contextual information may include any data that gives context to a particular description. In some embodiments, the contextual information includes text after a description of additional health record information. In some embodiments, the contextual information includes metadata of the examined document, a URL contained within the examined document, other text contained within the examined document, or a portion thereof. For each reference discovered in the health record, an attempt is made to retrieve the referred document or to extend the network in order to subsequently retrieve the document. Retrieved documents are added to the original document to extend the clinical record being created and provide a more complete record of patient care.

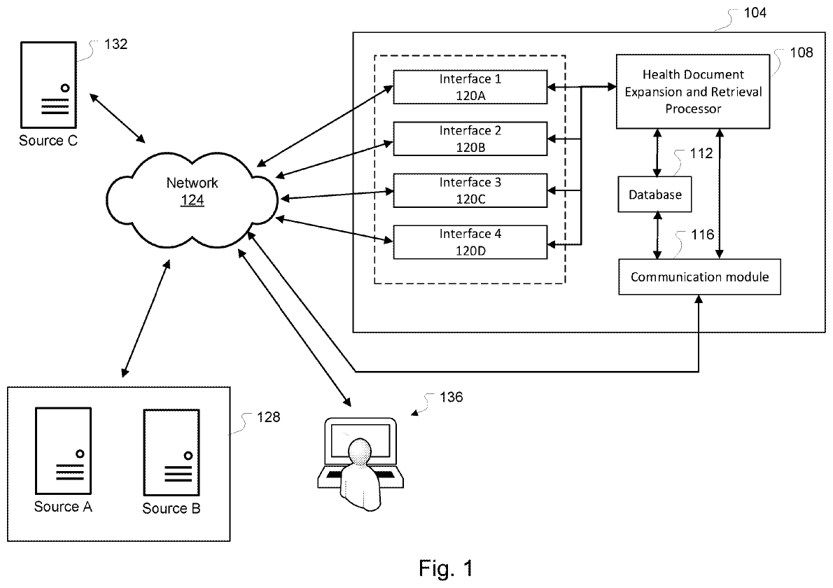

FIG. 1 depicts a health records network 100 in accordance with examples of the present disclosure. The health records network 100 may include a health document expansion retrieval system 104, one or more sources of health records 128 and 132, and one or more targets 136, also referred to as a requester. Each of the health document expansion retrieval system 104, one or more sources of health records 128 and/or 132, and one or more targets 136 may communication or otherwise be communicatively coupled to one another via the network 124. The network 124 may be without limitation, a Wide Area Network (WAN), such as the Internet, a Local Area Network (LAN), a Personal Area Network (PAN), a Public Switched Telephone Network (PSTN), a Plain Old Telephone Service (POTS) network, a cellular communications network, or combinations thereof. The Internet is an example of the communication network that constitutes an Internet Protocol (IP) network including many computers, computing networks, and other communication devices located all over the world, which are connected through many systems and other means. In one configuration, the telecommunication network is a public network supporting the TCP/IP suite of protocols. Communications supported by the telecommunication network include real-time, near-real-time, and non-real-time communications.

The health document expansion retrieval system 104 may include a health document expansion retrieval processor 108, one or more databases 112, one or more communication modules 116, and one or more user interfaces 120A-120D. In some examples, the health document expansion retrieval processor 108 may be a portal that provides functionalities for a point of access on the web or Internet. That is, as will be described below, a health document expansion retrieval processor 108 may provide centralized management capabilities for receiving a request for a health record and fulfilling such request by retrieving health record information from one or more sources, such as source 128, assembling a health record, and providing the assembled health record to one or more targets, such as target 136. Accordingly, the one or more targets 136 may interact with one or more of the user interfaces 120A-120D to provide a health record request to the health document expansion retrieval system 104 and receive the requested health records from the health document expansion retrieval system 104 via the one or more user interfaces 120A-120D.

The health document expansion retrieval system 104 may store one or more health records in the database 112. More specifically, the database may store information associated with health records gathered from the one or more sources 128 and/or 132, and may act as a repository of health record information. Moreover, as one or more health records are assembled from information gathered from the one or more sources 128 and/or 132, the health document expansion retrieval system 104 may store source identification information for locating and/or identifying additional and/or other sources of health record information.

As previously discussed, the health document expansion retrieval system 104 may be a portal. Accordingly, a management entity may manage access by the target, or requester 136. Moreover, one or more rules limiting the access of the requester and/or establishing eligibilities with respect to what type of health record information may be viewed by the requester. Moreover, the management entity may include a number of tools to configure, set up, and operate the portal. For example, the management entity may include a number of portlets customized for various users. It may include specialized processing subsystems or engines to process and generate one or more of the user interfaces 120A-120D.

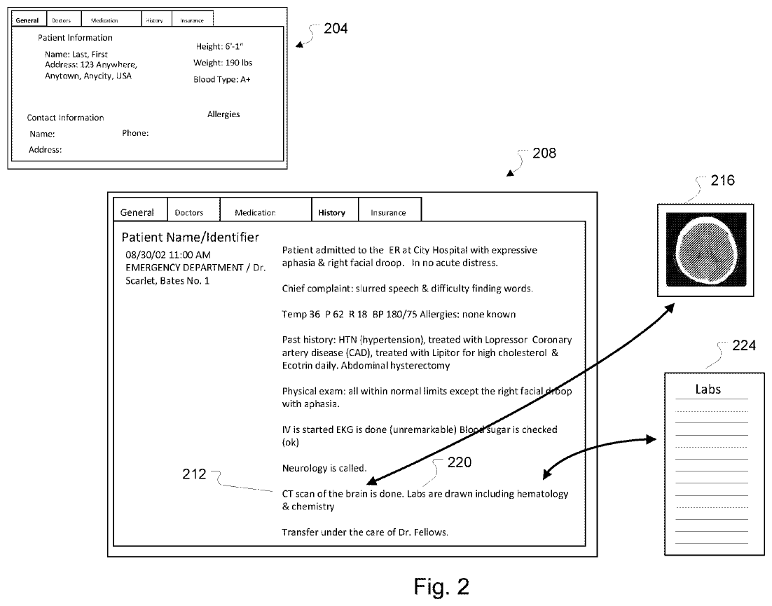

FIG. 2 depicts a health record 204 in accordance with examples of the present disclosure. The health record 204 may be a health record retrieved from one or more sources as previously discussed. In addition, the health record 204 depicted in FIG. 2 should not be considered limiting; that is, the health record 204 is an example health record; other health records, including varying types of health records (e.g., electronic, paper, or otherwise) are contemplated herein. Moreover, as health record 204 is an example of a health record, the actual health records retrieved from one or more sources may be different than that which is displayed in FIG. 2.

In addition, the health record 204 may include various organizations or partitions separating health record information. For example, a health record 204 may include a general portion which provides general information about a patient; a doctors portion which provides information about and/or to doctors associated with the patient; a medication portion which provides additional information about medications that have been and/or are prescribed to the patient; a history portion providing information about past treatments and/or care; and an insurance portion which may provide additional insurance information and/or billing related information. As the previously described portions are provide for example purposes only, a health record may include more or fewer portions than that which has been described above. The health record 204 may include a patient name and/or identifier, such as a reference number; the patient name and/or identifier may be utilized by the health document expansion retrieval system 104 to associate health record information with a specific or specified patient or individual.

As further depicted in FIG. 2, the health record 208 generally depicts an example history portion of the health record 204. As previously described, the health document expansion retrieval system 104 may analyze one or more portions of the health record, such as health record 208, and determine if the health record references or otherwise refers to additional health record information. For example, the history portion of the health record 208 may reference a CT scan at 212. The CT scan 212 may be hyperlinked to indicate a location associated with the CT scan 212 which includes additional CT scan information, such as the CT scan itself 216. As another example, the health document expansion retrieval system 104 may determine that the health record 208 references labs 220; labs 220 may or may not be hyperlinked or otherwise associated with a location identifying lab results 224. Accordingly, the health document expansion retrieval system 104 may utilize contextual information associated with the health record 208 to determine a location, such as a health record system, health record provider, hospital, care provider or otherwise, that may include the lab results 224 associated with the reference to the lab 220. The health document expansion retrieval system 104 may then interface with such system to retrieve the lab results 224 and store or otherwise associate the retrieved lab results 224 with the patient name/identifier and/or reference.

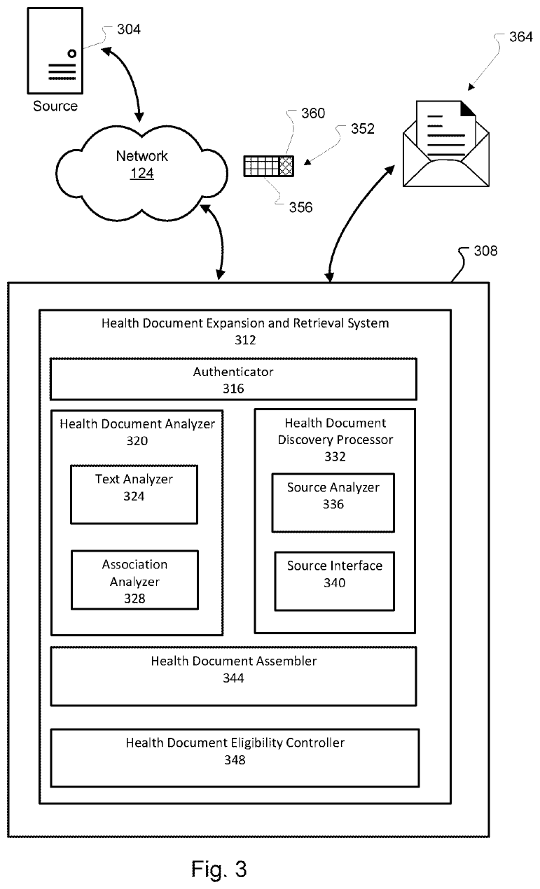

FIG. 3 depicts additional details of a health records system 308 including a health document expansion retrieval system 312. The health document expansion retrieval system 312 may be the same as or similar to the health document expansion retrieval system 104 as previously described and may operate and/or function in a similar manner. In accordance with at least one example of the present disclosure, a target, or requester, 304 may initiate or otherwise make a request to the health document expansion retrieval system 312 via the network 124; the health document expansion retrieval system 312 may initially authenticate the requester 304 and verify that the requester 304 has access rights to the health document expansion retrieval system 312 and a requested health record. Initially, the request from the requester 304 may be provided with a reference 360 included in a request 352 identifying one or more patients, and/or one or more pieces of health record information. The request may include the reference 360 and additional metadata 356 comprising one or more request parameters. For example, the requester 304 may request all available health records for patients having a specific condition, receiving a specific type of care, or being treated with a specific treatment protocol. In some examples, the requester 304 may request health record information utilizing a patient's name and/or reference number.

The health document expansion retrieval system 312 may receive the request 352 and authenticate the requester 304 utilizing the authenticator 316, and further retrieve health record information stored in a database 112 for example. The database 112 may previously have been populated with health record information for one or more patients. Accordingly, the health document analyzer 320 may analyze the health record information associated with one or more patients to determine if a health record is complete. For example, a text analyzer 324 may analyze text of the health record and parse or otherwise determine that a text portion may refer to a portion of the health record that is incomplete. As previously discussed for example, a portion of the text may refer to lab results, where the lab results are not part of the existing health record. Accordingly, the association analyzer may determine if any existing associations exist for the portion of text and/or a significance associated with the portion of text. That is, the text analyzer 324 may analyze text and the association analyzer 328 may utilize parsed text to determine what portion(s) of text are currently associated with additional health record information. For example, the database 112 may include a list of terms (e.g., “lab results,” “CT scan,” “prior visit,” etc.) that are indicative of additional health record information. The text analyzer 324 may parse through the text of stored health record information of a particular patient or text of a request for health record information in order to find hard or soft matches of one or more portions of the parsed text to one of the terms. Furthermore, the number or list of terms may be pre-populated and/or added to over time either by a user or via a machine learning algorithm. In another example, the text analyzer 324 may determine portions of text that are indicative of additional health record information by having multiple strings or regex patterns stored in the database 112 (or program memory) such that the text analyzer 324 may match portions of the parsed text (or the binary data associated with the parsed text) to the multiple strings or regex patterns. In some embodiments, the text analyzer 324 may be configured to connect to an external source (e.g., a centralized database or a database in the cloud) of keywords, terms, strings, or regex patterns via an application programming interface (API) and to compare the parsed text to the accessed keywords, terms, strings, or regex patterns in order to determine whether the text contains information indicative of additional or supplemental health record information.

In instances where the health document analyzer 320 determines that at least a portion of the health record is incomplete (e.g., that there is additional health record information that the health document expansion retrieval system does not have), the health document expansion retrieval system 312 may direct the health document discovery processor 332 to locate the missing health record information. Utilizing contextual information associated with the health record, the health document discovery processor 332 may utilize a source analyzer to locate one or more sources of health record information and more specifically, one or more sources of health record information associated with the patient and/or reference. As one example, a portion of a health record may indicate that a specific treatment was performed at a specific hospital or location; the source analyzer may utilize such contextual information to identify a health record system, or source, where such additional health record information may be located. Accordingly, the source interface 340 may utilize the hospital name for instance, and determine a health record system utilized by the hospital. In some instances, the health document expansion retrieval system 312 may have previously connected with the identified health record system of said hospital and may store such connection information, for example in the database 112; such connection or interface information may be utilized to connect to the health record system of the particular hospital for instance. In instances where connection information has not been established or is otherwise incomplete, the health document expansion retrieval system 312 may attempt to determine connection information from contextual information and/or proceed to a state where additional information is requested from one or more parties, by automatic and/or manual means.

In instances where the health document discovery processor 332 retrieves additional health record information, the retrieved health record information may be analyzed via the health document analyzer 320 and the process of identifying portions of the now retrieved health record may be repeated. That is, the health document expansion retrieval system 316 may operate in a recursive manner, such that subsequent retrieval of health record information is subjected to the same or similar processing as the initial document or health record information request. Accordingly, a health document assembler 344 may assemble the requested health record information into an electronic document for delivery to the requester 304 and/or for storage in the database 112 and associated with a patient identified via the reference. In some instances, the health document eligibility controller 348 may determine an eligibility of the requester 304 to view information contained in the new more complete health record. For example, the requester may not be eligible to view personal identifiable information and all personal identifiable information may be removed from the assembled health record. The assembled health record 364 may then be provided to the requester 304 for example via one or more interfaces 120A-120D.

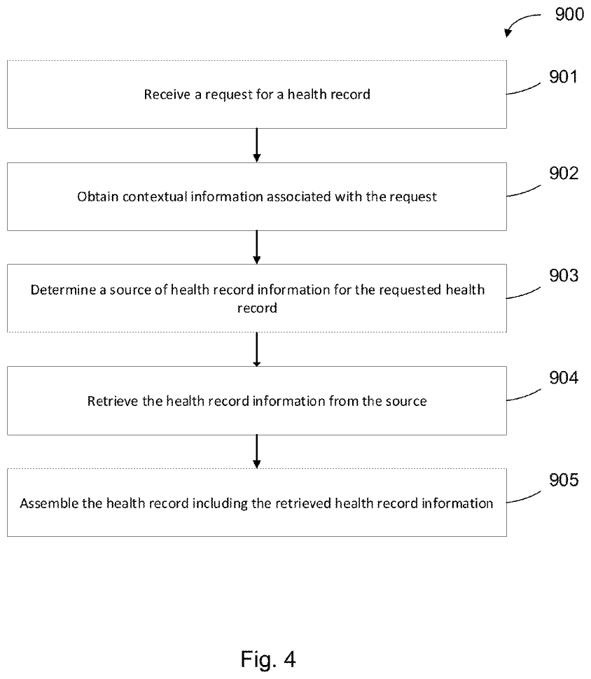

FIG. 4 depicts an example flow diagram of a method 900 of operations of a health document expansion and retrieval system. The health document expansion retrieval system receives a request for a health record at operation 901. Similar to operation 105 described below, in some embodiments, the request includes at least one document reference. The document reference may include a description of requested health record information, a description of a document containing health record information, and/or a location of the health record information or document.

Additionally or alternatively, as described in reference to the health document expansion retrieval system 312 of FIG. 3, in some embodiments, the request indicates requested health record information for a particular person (e.g., patient). In some embodiments, health record information for the particular person may be already stored in a database (e.g., database 112) of the health document expansion and retrieval system. As described in reference to FIG. 3, the health document expansion and retrieval system may automatically retrieve (or access) the health record information stored in the database and examine or analyze the text and associated data of the health record information to determine whether the health record information references additional health record information (e.g., references a document that contains additional health record information). In some embodiments, the examination or analysis of the portion of the health record information is similar to the processes described below in reference to operation 180.

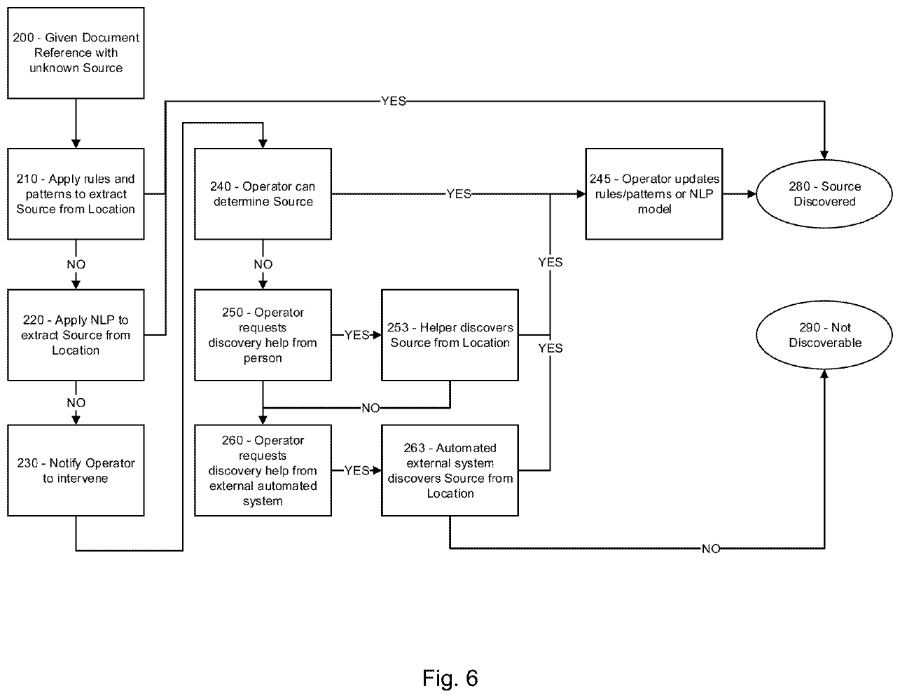

The health document expansion retrieval system obtains contextual information associated with the request at operation 902. In some embodiments, the document reference of the request may include a description of a document containing health record information. As described in reference to operation 105, the description of the document may include an implicit reference or explicit reference to health record information and/or the location of where the health record information may be stored. In some embodiments, as described below in reference to operation 130 and FIG. 6, the health document expansion and retrieval system utilizes the contextual information of the request to determine an electronic location of the requested health record information. For example, as explained in reference to FIG. 6, in some embodiments, the health document expansion and retrieval system parses the text of the request and pattern matches portions of the parsed text to determine an electronic location of the requested health record information. In some embodiments, the electronic location of the requested health record information corresponds to a source (a computing system of a health record provider) of the requested health record information. In some embodiments, the request may include a document reference that includes a particular field that explicitly references the source and electronic address of the source (e.g., and thereby the electronic location of the health record information on the network).

In some embodiments, as described above in reference to FIG. 3, the requested health record information may be automatically retrieved or accessed from the database 112 if the database includes health record information for a particular person identified in the request. In some embodiments, health document expansion and retrieval system may utilize natural language processing or parse the text of the request and pattern match the parsed text to determine the patient's name. That is, the health document expansion and retrieval system may parse the text of the request and pattern match the parsed text to names of patients that are stored within the database 112. In response to determining a name that corresponds to a name in the database 112 having stored health record information, the health document expansion and retrieval system may automatically access the health record information.

The health document expansion retrieval system determines that health record information is to be retrieved from a health record provider at operation 903. In some embodiments, the health document expansion and retrieval system utilizes natural language processing to process text of the request and cross reference portions of the text (e.g., contextual information) to sources stored in the database 112 in order to determine the source that likely contains the requested health record information. In some embodiments, the database 112 is updated each time a source is determined to include an identification of a computing system (e.g., identification or address on the network) of the particular custodian and a name of the custodian of health record information.

In an embodiment where the health document expansion retrieval system has automatically accessed or retrieved health record information for the patient indicated by the request, the health document expansion retrieval system may determine additional health record information that is to be retrieved from a health record provider. Similar to operation 180 described below, the health document expansion and retrieval system may parse through the text of the health record information and pattern match portions of the parsed text to the description contained in the document reference of the request to determine if there is additional health record information that is not contained within the health record information stored in the database 112. The health document expansion and retrieval system may then use contextual information of the health record information stored in the database 112 to determine an electronic location and/or description of the additional health record information. As described in reference to FIG. 3, the contextual information may include an explicit reference to a particular document and source containing the additional health record information or an implicit reference to a document and/or source containing the additional health record information.

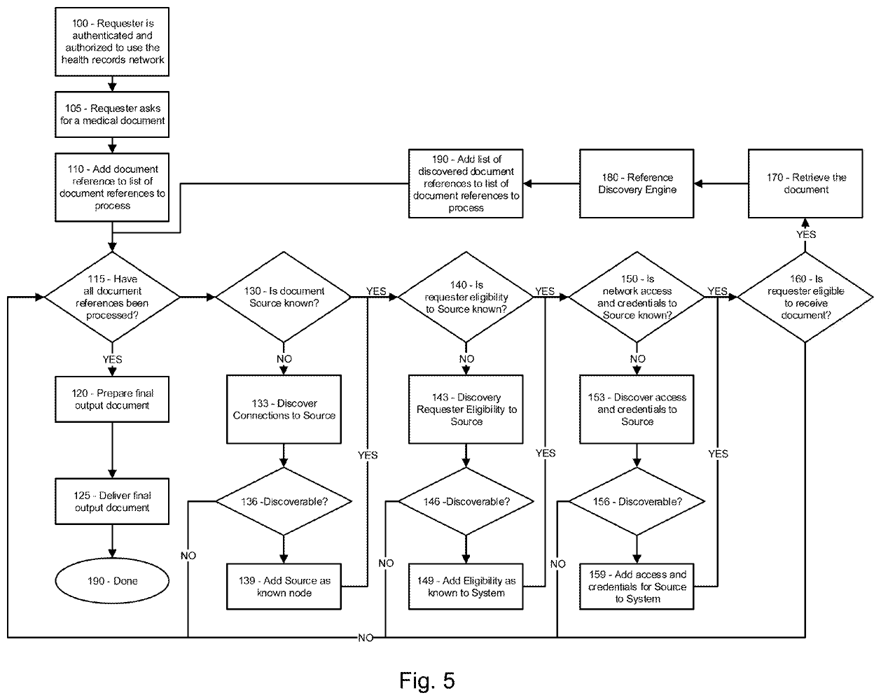

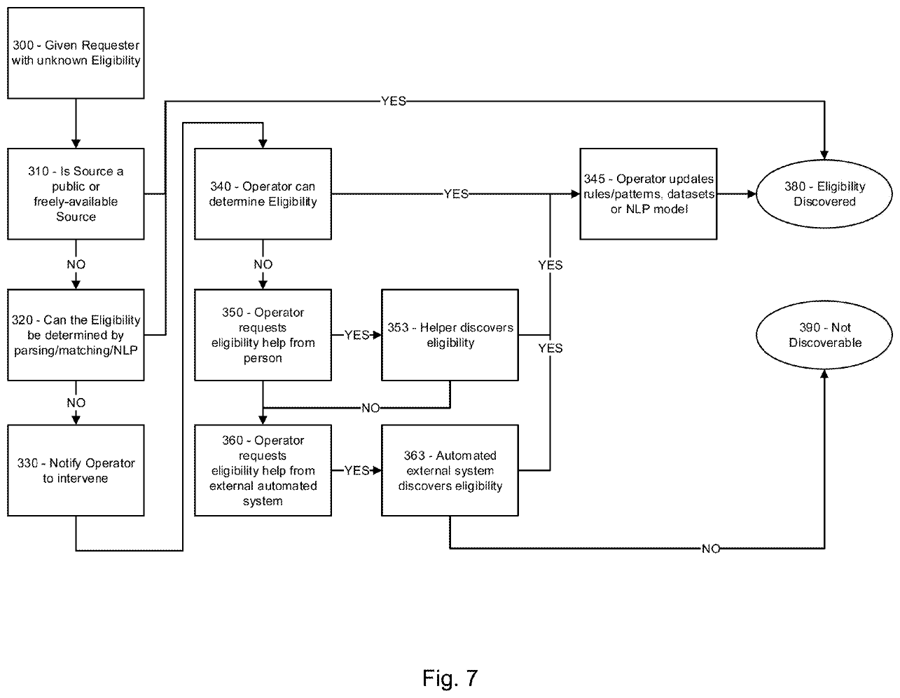

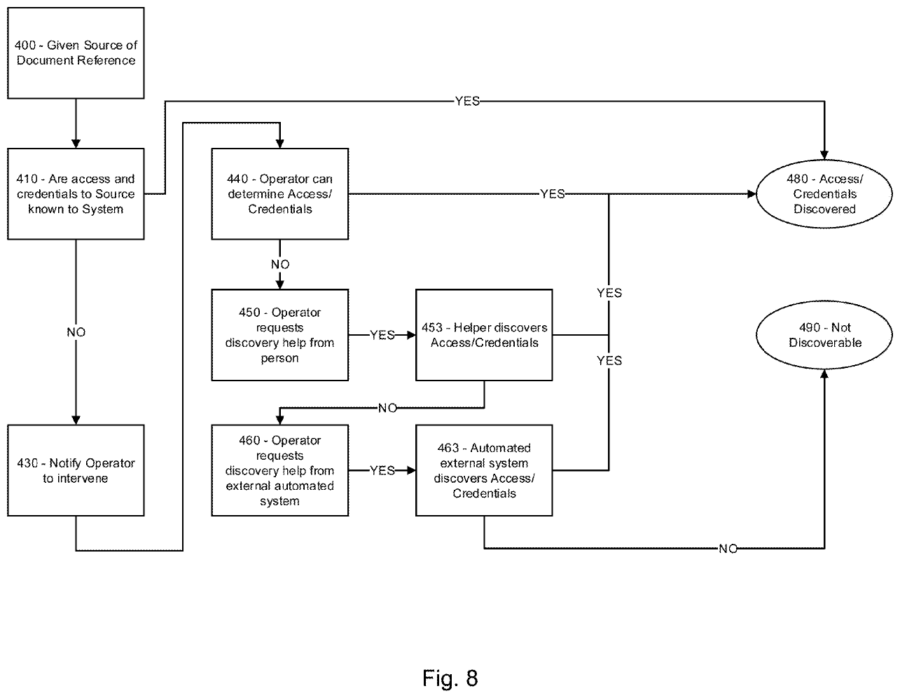

The health document expansion and retrieval system retrieves the health record information from the health record provider at operation 904. In some embodiments, as described below in reference to FIG. 5, the health document expansion and retrieval system uses the identified source (e.g., electronic location corresponding to an address of the source on the network) to retrieve the health record information or additional health record information. In some embodiments, as described below in reference to operation 140 and FIG. 7, the health document expansion and retrieval system may determines the eligibility of the requestor to access information from the source (e.g., health record provider). As described below, in some embodiments, the eligibility of the requestor (e.g., and multiple requestors) may be stored in the database 112. In some embodiments, as described below in reference to operation 150 and FIG. 8, the health document expansion and retrieval system may determine how to interface with the source (e.g., computing system of the health record provider). For example, the health document expansion and retrieval system may determine a proper communication method (e.g., via an application programming interface, URL, online portal, etc.) and the necessary network credentials to access or interface with the source (e.g., computing system of the health record provider). In some embodiments, the network credentials are stored within the database 112). As described below in reference to operation 160, in some embodiments, the health document expansion and retrieval system may establish a connection with the computing system of the health record provider and request the health record information or additional health record information. In some embodiments, the request to the health record provider may indicate a name of the patient, the date on which the patient visited the health record provider, description of the requested health record information, or a combination thereof. The health record provider may then transmit the health record information or allow the health document expansion and retrieval system to access the health record information directly from a database of the health record provider.

The health document expansion and retrieval system assembles the health record including the health record information at operation 905. In some embodiments, the health record is also assembled using the additional health record information retrieved by the health document expansion and retrieval system. In some embodiments, a portion of the health record information is redacted based on rules governing the information that the requester may receive, as described below in reference to operation 120. In some embodiments, health document expansion and retrieval system may then electronically provide the health record to the requestor.

FIG. 5 depicts an example flow of operations of the health document expansion retrieval system. When a requesting entity begins to interact with the health records network, a right to access the network is tested at operation 100. The requester is authenticated, for example by common internet authentication methods like OAuth or Basic Encryption, or other authentication methods. The requester is subsequently authorized for their rights and roles on the network, which may be based on commercial arrangements with the network provider, such as subscription tier and account status or based on governmental rules supporting patient access to healthcare data. If the requester is properly authenticated and authorized to make a request, the workflow proceeds to operation 105.

At operation 105, the requester asks for a particular health records document. The health record document may be a document applying to one patient over a given time period or for a given type of medical procedure or a set of encounters linked to an event or health condition. Alternatively, or in addition, the health record document may be a summary document related to use of a medical facility on a given day. Further, the health record document may be a document of billing information related to a number of insurance claims. The health record document may be described by the requester with a “document reference,” consisting of a description of the document and a location of the document in a given source.

The document reference may be added to an initially empty list of document references that are to be processed by the system, at operation 110. At operation 115, the health document expansion retrieval system checks to see if there are any document references that have not yet been processed. Initially, there will be one, so flow proceeds to operation 130.

When all document references in the list have been processed, then the health document expansion retrieval system proceeds to operation 120 to prepare the final document for delivery to the requester. During this document preparation process, all documents discovered by the system are collated into a final assembled document. In addition, eligibility rules that apply to the requester for the referenced documents, in relationship to the source of each document, are applied to each part of the assembled document such that the document provided to the requester complies with the one or more eligibility rules. Eligibility rules may be derived from commercial arrangements between parties in the network, based on the use-case for the requested document and/or based on governmental rules supporting patient access to healthcare data. Thus, if there are to be multiple recipients of the requested data, a version of the final document may be prepared based on the corresponding eligibility rules for a particular recipient. In some instances, eligibility information may reside in a system external to the data repository. For example, if a given requester is eligible to receive documents, but only if the data within a document does not contain personally identifiable data (PII), then the assembled document prepared by the system will not include the PII in the final document prepared for the requester. In another example, a requester may not be eligible to receive billing information from the source of the “document reference.” Once the final document has been prepared with all available discovered documents and according to the eligibility rules, the assembled document is delivered to the recipient at the target node at operation 125. The flow for the request is thus completed at operation 190. When the system seeks to retrieve a document at a document reference (description and location on a source), there may be additional tests that occur at operations 130, 140 and 150 as described herein.

Further, a document reference contains a document description and a location on the network. The description and the location may be either structured digital information or unstructured data, in a well-known data format or not. For example, the description may be a filename or other identifiers, such as patient name, location of service, attending physician. Explicit references to computer system file paths or URI are identified and added to the list of discovered references for this document. These explicit references identify a location for a referenced document. Inferred references to other documents, such as a mention of a radiology image for the patient contained in physician's visit notes, when there is no such image explicitly referenced or attached to this record, can be discovered by the health document analyzer, using techniques which may include Natural Language Processing. Thus, these inferred references identify the location of a referenced document. The locations of the referenced documents exist as nodes that may already be known to the health records network or may be previously unknown locations. The location may be, though not limited to, a file system, a proprietary records system, a content management system, a database, a website, a network-accessible API. The reference may be complete, as the complete file path to a document on a file system, or may be incomplete, like the base URL of a FHIR server that contains the referenced document.

At operation 130, the health document expansion retrieval system determines if the source of the document location is known to the health document expansion retrieval system. A database may be used to store the known relationships between the health records network and the particular data sources that contain locations of documents. If the source associated with the location is known to the system, then the workflow proceeds to operation 140. Alternatively, if the source associated with the location of the document is not known to the system, then the Source Discovery process is invoked at operation 133. If the Source Discovery Process is successful, as tested in operation 136, then the key information about the data source node is recorded in the records of the system at operation 139 and the workflow proceeds to operation 140. For example, the key information may be stored in the database 112. If the Source Discovery Process is unable to obtain enough information about the source node, then the document reference is considered undiscoverable, is marked as processed and the workflow returns to operation 115. Alternatively, or in addition, when the Source Discovery Process cannot discover enough information about the source node, the undiscoverable document reference may be queued and then passed on to a secondary process (manual, automatic or mixed), which allows the complete document reference to be located in order to add the novel location to the system, outside of the context of completing the requested document.

At operation 133, if the document source for the locations is not known to the health document expansion retrieval system, then the health document expansion retrieval system attempts to determine the novel source from the location information. The location may include but is not limited to a URL for a FHIR service API call, from which the source node can easily be extracted. The location may include but is not limited to a UNC file path on the file sharing network in a data custodian's digital infrastructure. The location may include but is not limited to a plain text description such as “at the Emergency Room of the Community Hospital in New York City”. If the previously unknown source of the data can be extracted from the location in the document reference by digital parsing and pattern matching rules, or by the application of machine learning techniques like natural language processing (NLP), then the source node is considered discovered and the workflow proceeds from operation 136 to operation 139.

If the system cannot digitally infer the source node from the location, then the location and document reference may be added to a work queue for a human operator, who may use a system that includes a graphical user interface, to continue the source discovery process, by one or more means of hybrid machine and human interactions for example. Additional details of the workflow at operation 133 is provided in FIG. 6 and the corresponding description.

At operation 200, the operation 133 workflow begins, when a document reference, consisting of a document description and a document location, are passed to the Source Discovery process. At operation 210, the system applies patterns and rules known to the system to apply parsing and matching to the Location data in order to discover the Source. For example, if the document reference gives a location of a FHIR API endpoint such as “https://fhir.example.com/Patient/111” then a rule for discovering FHIR endpoints from locations may match on the sub-pattern “fhir.*.com” and derive a Source of “https://fhir.example.com”. In another example, if the Location information includes the Tax Identification Number (TIN) of a health system, which matches the pattern of two digits, a dash, then seven digits, such as 12-3456789, the matching rules may determine that there is a TIN in the location. If the system has access table to a correlation table of TINs and Sources, such as a mapping of TINs to health system EMRs, then the data Source can be derived from the TIN data in the location. The above examples are for the purpose of illustration and are not meant to limit the scope of the examples which may utilize a broad range of external databases and indexes to support the process.

If the source is discovered, the workflow proceeds to operation 280 with a discoverable Source. If the rules and patterns applied at operation 210 cannot discover the Source, then then Location can be passed to a natural language processing (NLP) or other artificial intelligence analyzer at operation 220. In instances where the location is unstructured, for example, where the Location is unstructured, as when the Reference Discovery Processor or original requester specified a document reference with a description of “electrocardiogram on Dec. 12, 2017” with a location of “Seen by Dr. Jones at Comm. Hosp. of Central City ER”. The NLP analyzer may be trained to determine that the location phrase references “Community Hospital of Central City Emergency Room”, from which the health document expansion retrieval system can derive that the Source is in the EMR used by the emergency department at Community Hospital of Central City.

If the source is discovered, the workflow proceeds to operation 280 with a discoverable Source. If the system cannot discover the Source at operation 220, then a human operator may be notified to intervene at operation 230. The operator may be notified by one or more electronic means, including, but not limited to, email, FAX, SMS, notification on a messaging platform such as Slack, IRC, and/or voicemail. The operator may be directed to use a human-computer interface, such as a graphical user interface (GUI), to attempt to discover the Source of the referenced document.

At operation 240, if the Operator may determine the Source without further assistance, then the workflow proceeds to operation 245. At operation 245, the Operator may enhance the system by using the GUI or other means to update and save changes to the rules and patterns and/or NLP model, based on new understandings gained by the discovery of the Source from the Location. For example, the Operator may have deduced a new pattern for matching the Source identified in a structured Location field or the Operator may submit new training data to the NLP system to enhance the NLP model. After operation 245, the workflow proceeds to operation 280 with a discoverable Source.

If the Operator cannot determine the Source without help, at operation 250 the Operator may request help from another operator. The health document expansion retrieval system allows the Operator to request help in discovering the Source from the Location by contacting one or more entities by one or more means, including but not limited to email, FAX, SMS, notification on a messaging platform like Slack or IRC, postal mail or voicemail. The Operator uses the GUI or other interface to record the request. The helping entity can be directed to provide their helpful answer by a variety of means. These include submitting the information back to the system directly through a digital form, conveyed by email, a web portal or other means of entering the Source information into the System directly. Alternately, the Operator may receive the information from the helpful entity and enter it into the system themselves. If the Source has been discovered, workflow may proceed to operation 245. If the Operator is unable to discover the Source with human help, then the workflow proceeds to operation 260.

The system may be configured to contact one or more automated information systems to request help in during the discovery of the Source from the Location information. The system may utilize use a digital interface, whether a modern RESTful interface or other means including but not limited to proprietary RCP protocols, various TCP/IP protocols, or web services like SOAP. The Operator may use the system to make the request to the external systems. The external systems may be able to provide a response that the health document expansion retrieval system can ingest data utilizing one or more interfaces. In examples where results from the external system need to be interpreted by an Operator, the Operator may utilize the human-computer interface to add the discovered Source to the system. If the Source has been discovered, the workflow proceeds to operation 245. If not, the workflow has failed to discover the Source and proceeds to operation 290 with a non-discoverable Source. In some examples, there may be parameters that limit the amount of time spent in any operation or aggregation of processes before the system marks the Source as non-discoverable and proceeds to operation 290.

After the Source Discovery workflow is successfully completed, the main workflow proceeds to operation 140, to determine if the Requester is eligible to receive the referenced document. At operation 140, the health document expansion retrieval system determines if the eligibility of the Requester to retrieve documents from the Source of the document reference is known to the system. A database, such as database 112, may be used to store the known relationships between the requesters of data and the particular data sources that contain locations of documents. If the eligibility relationship between the requester and the source associated with the document reference is known to the system, then the workflow proceeds to operation 150. If the system does not yet know the eligibility relationship, then the requester and document reference are added to a work queue for a human operator, who uses a system that includes a graphical user interface, to continue the eligibility discovery process, by one or more means of hybrid machine and human interactions. Additional details directed to operation 143 is provided with respect to FIG. 6 and the corresponding description.

At operation 300, the operation 143 workflow begins, when information about the requesting entity and the document reference are passed to the Eligibility Discovery process. At operation 310, the health document expansion retrieval system reviews data known the system, as stored in databases or dynamically available, as from an online data registry, to determine if the Source is a public or other freely available data source, where the Requester has implicit eligibility to the data on the Source, because of the open nature the data source. For example, the Centers for Medicare and Medicaid maintain a public data source for records related to healthcare providers who have patients covered by Medicare and Medicaid programs. The eligibility for requesters to this data source is implicitly known to be open, due to the nature of the data source. If the system has determined that the eligibility information has been discovered because of the open nature of the source, then the workflow continues to operation 380.

If the eligibility is not discovered in operation 310, the workflow continues to operation 320. At operation 320, the health document expansion retrieval system reviews data known the system, in some cases as stored in databases or dynamically available, as from an online data registry, to determine if eligibility data for the requester at the data source is known. This is not a test for the eligibility for the document to be retrieved, but for the knowledge of eligibility information for the requester-source relationship, for the type of data generally requested in the document reference. The system may apply patterns and rules known to the system to apply parsing and matching to the information about the Requester and/or the Source in order to discover the existence of the eligibility information. Likewise, the health document expansion retrieval system may use a natural language processing (NLP) or other artificial intelligence processor to help determine if the eligibility information is known to the system by deducing the relationship between the Requester and the Source and comparing that to eligibility information known to the system. If the eligibility relationship has been discovered in this process, then the workflow continues to operation 380. If the system cannot discover the eligibility information at operation 320, then a human operator may be notified to intervene at operation 330. The operator may be notified by one or more electronic means, including, but not limited to, e-mail, FAX, SMS, notification on a messaging platform like Slack, IRC, and/or or voicemail. The operator may then be directed to use a human-computer interface, such as a graphical user interface (GUI), to attempt to discover the eligibility information for the Requester-Source relationship.

At operation 340, if the Operator can determine the eligibility without further assistance, then the workflow proceeds to operation 345. In operation 345, the Operator may enhance the system by using the GUI or other means to update and save changes to the rules and patterns, the system's records for freely available and public sources, external eligibility resource and/or NLP model, based on new understandings gained by the discovery of the eligibility information. For example, the Operator may have discovered a previously unknown public data source from the referenced document. After operation 345, the workflow proceeds to operation 380 with a discoverable eligibility. If the Operator cannot determine the eligibility without help, at operation 350 the Operator can request help from another operator or entity. The health document expansion retrieval system allows the Operator to request help in discovering the Source from the Location by contacting one or more entities by one or more means, including but not limited to e-mail, FAX, SMS, notification on a messaging platform like Slack, IRC, postal mail, and/or voicemail. The Operator may utilize the GUI or other interface to record the request. The helping entity can be directed to provide their helpful answer by a variety of means. These include submitting the information back to the health document expansion retrieval system directly through a digital form, conveyed by email, a web portal, and/or other means of entering the eligibility information into the health document expansion retrieval system directly. Alternately, or in addition, the Operator may receive the answer from the helpful entity and enter it into the system themselves. If the eligibility information has been discovered, the workflow proceeds to operation 345. If the Operator cannot discover the eligibility information with additional help, then the workflow proceeds to operation 360.

The system may be configured to contact one or more automated information systems to request help in the discovery of the eligibility information for the Requester-Source relationship. The health document expansion retrieval system may utilize a digital interface, whether a modern RESTful interface or other means including but not limited to proprietary RCP protocols, various TCP/IP protocols, or web services like SOAP. The Operator may utilize the health document expansion retrieval system to make the request to external systems. The external systems may be able to provide a response indicating that the health document expansion retrieval system may ingest digitally, in the case when the system and the external automated system can be interfaced.

In the case where the results from the external system are to be interpreted by the Operator, the Operator may use the human-computer interface to add the discovered eligibility information to the system. If the eligibility information has been discovered, the workflow proceeds to operation 345. If not, the workflow has failed to discover the Source and proceeds to operation 390 with a non-discoverable eligibility. In examples, there may be parameters that limit the amount of time spent in any process or aggregation of processes before the system marks the Eligibility as non-discoverable and proceed to operation 390.

After the Eligibility Discovery workflow is successfully completed, the main workflow proceeds to operation 150, to determine if the health records network system has access to and the credentials required to receive the document reference from the source. At operation 150, the system determines if the requester has the capability, through access and credentials, to retrieve the document reference from the source. A database may be used to store the known access parameters and credentials the particular data sources known to the system. If the access parameters and credentials between the system and the source associated with the document reference is known to the system, then the workflow proceeds to operation 160.

If the health document expansion retrieval system does not yet have the access parameters and credentials, then the source information and document reference are added to a work queue for a human operator, who uses a system that includes a graphical user interface, to continue the eligibility discovery process, by one or more means of hybrid machine and human interactions. Additional details of operation 153 are provided with respect to FIG. 7 and the corresponding description.

At operation 400, the operation 153 workflow begins, when information about the Source and the document reference are passed to the Access/Credentials Discovery process. At operation 410, the health document expansion retrieval system reviews data known to the system, as stored in one or more databases 112 for example, to determine if the health document expansion retrieval system has sufficient information, connections and credentials to access the Source containing the document reference. If the system has determined that the information has been discovered because it is already known to the system, then the workflow continues to operation 480. If the access information and credentials are not discovered in operation 410, the workflow continues to operation 430 and a human operator is notified to intervene in the process. The operator is notified by one or more electronic means, including, but not limited to, e-mail, FAX, SMS, notification on a messaging platform like Slack, IRC, and/or voicemail. The operator may be directed to use a human-computer interface, such as a graphical user interface (GUI), to attempt to discover the eligibility information for the Requester-Source relationship.

At operation 440, if the Operator can determine the eligibility without further assistance, then the workflow proceeds to operation 480. If the Operator cannot determine the eligibility without help, at operation 450 the Operator can request help from another Operator or entity. The system allows the Operator to request help in discovering the access information, connections and credentials by contacting one or more entities by one or more means, including but not limited to e-mail, FAX, SMS, notification on a messaging platform like Slack, IRC, postal mail, and/or voicemail. The Operator uses the GUI or other interface to record the request. The helping entity may be directed to provide information by a variety of means. These include but are not limited to submitting the information back to the health document expansion retrieval system directly through a digital form, conveyed by email, a web portal or other means of entering the eligibility information into the System directly. Alternately, the Operator may receive the information from the helpful entity and enter it into the system themselves. If the access information, connections and credentials have been discovered, the workflow proceeds to operation 480. If the Operator cannot discover the information with human help, then the workflow proceeds to operation 460.

The health document expansion retrieval system may be configured to contact one or more automated information systems to request help in the discovery of the access information, connections and credentials. The health document expansion retrieval system may use a digital interface, whether a modern RESTful interface or other means including but not limited to proprietary RCP protocols, various TCP/IP protocols, or web services like SOAP. The Operator may use the health document expansion retrieval system to make the request to the external systems. The external systems may be able to provide a response that the health document expansion retrieval system can ingest digitally, in examples where the health document expansion retrieval system and the external automated system interface with one another. In examples where the results from the external system need to be interpreted by the Operator, the Operator may use the human-computer interface to add the discovered access information, connections and credentials to the health document expansion retrieval system. If the information has been discovered, the workflow proceeds to operation 480. If not, the workflow has failed to discover the access information, connections and credentials and proceeds to operation 490 with a non-discoverable status.

In accordance with some examples of the present disclosure, the access and credentials allow automatic digital retrieval of documents. Alternatively, or in addition, access and credentials may support a manual document retrieval workflow, for example, for FAX-based documentation request and retrieval. In some examples of Access/Credentials Discovery workflow, there may be parameters that limit the amount of time spent in any process or aggregation of processes before the system marks the Access/Credentials as non-discoverable and proceeds to operation 490.

Subsequent to the Access/Credentials Discovery workflow being successfully completed, the main workflow proceeds to operation 160, to determine if the Requester is eligible to receive the particular document identified in the document reference. Thus, the health document expansion retrieval system may determine the eligibility of the requester to receive the document and all of the information required to try to retrieve the document from the source based on all information available to the health document expansion retrieval system.

The health document expansion retrieval system determines the eligibility of the Requester to receive the document references based on business and legal agreements between the requesting entity and the entity that is the custodian of the data in the Source. These agreements to access to healthcare records may be based on a number of factors. For example, one factor may be the purpose for which the document references is intended. That is, a Requester A may be entitled to receive medical billing information from Source B for the purpose of validating medical insurance claims but may not be entitled to receive the same records for insurance underwriting or for actuarial risk appraisal. Another factor in the eligibility arrangement between a health plan and a healthcare provider is memberships in active health plans during the period of service when a healthcare encounter took place at the health care provider. That is, the health plan Requester may only be eligible to document references when the document relates to a patient who received healthcare at the health provider at a time when the patient was an active member of an authorized insurance plan. In some examples, eligibility may require that the patient is attributed to the health provider system or individual practitioner providing services to the patient. That is, patient attribution expresses the relationship of a patient to a primary healthcare provider. In other cases, eligibility may be derived for governmental regulations or laws allowing patients to access their healthcare data.

In some examples, an arrangement between a requester and source entities can be conveyed in business rules that can be managed by the health document expansion retrieval system. This may include rules relating acceptable or forbidden purposes of use for a given Requester and Source, or for a multiplicity of Requesters or Sources. The Eligibility of plan members may be determined, in one exemplar, by comparing tables of members and their relevant identifiers (names, dates of birth, identification numbers with the requester and the source systems) and active dates of plan coverage against the date of service provided to the patient contained in the document reference. In another exemplar, tables of patient attribution can be examined to determine if the patient was attributed to the healthcare provider at the date of service provided to the patient contained in the document reference. Other eligibility checks may be based on document type, for example, image files may be restricted from eligibility to a given Requester. These examples are provided for illustrative purposes and are not a complete set of eligibility checks, which may encompass a set of any agreements between requester and the custodian of the source.

When all of the rules for eligibility in the agreement between requester and custodian of the source have been checked and the Requester is determined to be eligible to receive the health record information associated with the document reference, then workflow proceeds to operation 170, where the health document expansion retrieval system may attempt to retrieve the document from the Source. In some examples, the order of operation 160 and operation 170 may be reversed and may result in an efficiency improvement. operation 170 in the main workflow attempts to retrieve a healthcare document described by the document reference. When the location of the document reference is a known and the source is a complete and accessible source node on the health records network, the document is retrieved and the workflow moves to operation 180.

In some examples, document search, request and retrieval are automated digital processes. Alternatively, or in addition, the search for the document, the request for the document and the retrieval of the document from the source may include manual intervention, for example requests made by FAX, email, and/or telephone and document retrieval by email attachment and/or FAX. In such examples, a system operator may be notified that an action needs to occur and uses a computer-human interaction, as by use of a GUI, to transact a search, request, and retrieval process. The source may return the requested document to a subsystem that enters information into the health document expansion retrieval system directly. Alternately, the Operator may receive the document from the source and enter it into the system themselves.

If a document or document portion retrieved is in an image format, the health document expansion retrieval system may process it through one or more optical character recognition processors (OCR) to extract textual information. Computer vision techniques may also be used to extract textual information. If the document or document portion retrieved is an audio file (e.g. voice recording of a physician's encounter notes), then the health document expansion retrieval system may pass it through one or more Audio-to-Text processors to extract textual information. The extracted texts and the original document may be considered together through the remaining workflow.

Any document metadata may be considered together with the original retrieved document and any extracted texts through the workflow. This includes the provenance of the data, whether explicit or inferred. Operation 180 may use the Reference Discovery Process to build the list of all healthcare documents referenced in the retrieved document. Once a digitized healthcare document, whether a message, a voice recording, a video, a radiograph, a lab result, a medical order, composite data, summary document or other document has been extracted from the source system, then this digital health record is then analyzed by one or more analysis engines, such as the health document analyzer, to identify related healthcare documents and their sources or inferences to the existence of health records sources where documents may be found, whether digital or non-digital. The analysis engine looks through each element in the document, whether they represent discrete data (e.g. body temperature in degrees Celsius) or unstructured data (e.g. physicians notes, test results, discharge summaries, case notes, patient narratives, . . . ) or document metadata (e.g. at what time, on what system, by whom was a file generated, with what filename, etc.). In some examples, the system will extract inferred document sources as an incomplete document reference with an empty document description, as this can usefully expand the scope of the health records network. When the entire received document has been scanned by the analysis engines in the Reference Discovery Process, the list of discovered document references is added to the list of references and the workflow returns to operation 115. When entitled to a supplementary document, these additional document(s) may be retrieved digitally, as from an EMR, or manually, in which case they need to be converted into an electronic record by a document digitizer, as in the case of the primary document. Supplementary documents are also submitted to the analysis engine to discover additional inferred documents and repositories.

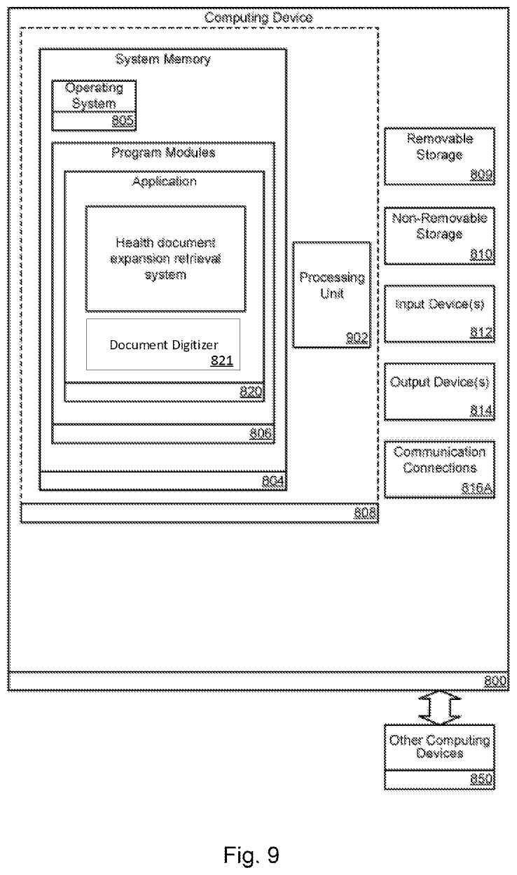

FIG. 9 is a block diagram illustrating physical components (e.g., hardware) of a computing device 800 with which aspects of the disclosure may be practiced. The computing device components described below may be suitable for the computing devices, such as the health document expansion retrieval system, and/or one or more endpoints associated with the requester and/or one or more sources. In a basic configuration, the computing device 800 may include at least one processing unit 802 and a system memory 804. Depending on the configuration and type of computing device, the system memory 804 may comprise, but is not limited to, volatile storage (e.g., random access memory), non-volatile storage (e.g., read-only memory), flash memory, or any combination of such memories. The system memory 804 may include an operating system 805 and one or more program modules 806 suitable for performing the various aspects disclosed herein such as the authenticator, the health document analyzer, the health document source discovery processors, document digitizer 821, the health document assembler, the health document eligibility controller, and one or more workflows associated with FIGS. 4-8. The operating system 805, for example, may be suitable for controlling the operation of the computing device 800. Furthermore, aspects of the disclosure may be practiced in conjunction with a graphics library, other operating systems, or any other application program and is not limited to any particular application or system. This basic configuration is illustrated in FIG. 9 by those components within a dashed line 808. The computing device 800 may have additional features or functionality. For example, the computing device 800 may also include additional data storage devices (removable and/or non-removable) such as, for example, magnetic disks, optical disks, or tape. Such additional storage is illustrated in FIG. 9 by a removable storage device 809 and a non-removable storage device 810.

As stated above, a number of program modules and data files may be stored in the system memory 804. While executing on the processing unit 802, the program modules 806 (e.g., one or more applications 820) may perform processes including, but not limited to, the aspects, as described herein. For example, the one or more applications 820 may include a document digitizer 821 configured to digitize documents that are manually input, received, or accessed by the health document expansion and retrieval system. As one example, as explained above, a particular document may be requested from a source that does not have the document stored in electronic form, an operator associated with the source may fax, scan, or otherwise upload the document into electronic form and transmit the document to the health document expansion and retrieval system. The document digitizer 821 may perform operations to digitize the document and thereby make the document readable by a computing device, processor, etc. (e.g., make the document available to be pattern matched or text of the document to be parsed). In some embodiments, the operations to digitize the document may include an optical character recognition program or other character recognition program or algorithm. In other embodiments document digitizer 821 may be separate from the one or more applications 820. Other program modules that may be used in accordance with aspects of the present disclosure may include electronic mail and contacts applications, word processing applications, spreadsheet applications, database applications, slide presentation applications, drawing or computer-aided application programs, etc.

Furthermore, aspects of the disclosure may be practiced in an electrical circuit comprising discrete electronic elements, packaged or integrated electronic chips containing logic gates, a circuit utilizing a microprocessor, or on a single chip containing electronic elements or microprocessors. For example, aspects of the disclosure may be practiced via a system-on-a-chip (SOC) where each or many of the components illustrated in FIG. 9 may be integrated onto a single integrated circuit. Such an SOC device may include one or more processing units, graphics units, communications units, system virtualization units and various application functionality all of which are integrated (or “burned”) onto the chip substrate as a single integrated circuit. When operating via an SOC, the functionality, described herein, with respect to the capability of client to switch protocols may be operated via application-specific logic integrated with other components of the computing device 800 on the single integrated circuit (chip). Aspects of the disclosure may also be practiced using other technologies capable of performing logical operations such as, for example, AND, OR, and NOT, including but not limited to mechanical, optical, fluidic, and quantum technologies. In addition, aspects of the disclosure may be practiced within a general purpose computer or in any other circuits or systems.

The computing device 800 may also have one or more input device(s) 812 such as a keyboard, a mouse, a pen, a sound or voice input device, a touch or swipe input device, etc. The output device(s) 814 such as a display, speakers, a printer, etc. may also be included. The aforementioned devices are examples and others may be used. The computing device 800 may include one or more communication connections 816A allowing communications with other computing devices 850. Examples of suitable communication connections 816A include, but are not limited to, radio frequency (RF) transmitter, receiver, and/or transceiver circuitry; universal serial bus (USB), parallel, network interface card, and/or serial ports.

The term computer readable media as used herein may include computer storage media. Computer storage media may include volatile and nonvolatile, removable and non-removable media implemented in any method or technology for storage of information, such as computer readable instructions, data structures, or program modules. The system memory 804, the removable storage device 809, and the non-removable storage device 810 are all computer storage media examples (e.g., memory storage). Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the computing device 800. Any such computer storage media may be part of the computing device 800. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

In the foregoing description, for the purpose of illustration, methods were described in a particular order. It should be appreciated that in alternate embodiments, the methods may be performed in a different order than that described. It should also be appreciated that the methods described above may be performed by hardware components or may be embodied in sequences of machine-executable instructions, which may be used to cause a machine, such as a general-purpose or special-purpose processor (GPU or CPU) or logic circuits programmed with the instructions to perform the methods (FPGA). These machine-executable instructions may be stored on one or more machine-readable mediums, such as CD-ROMs or other type of optical disks, floppy diskettes, ROMs, RAMs, EPROMs, EEPROMs, magnetic or optical cards, flash memory, or other types of machine-readable mediums suitable for storing electronic instructions. Alternatively, the methods may be performed by a combination of hardware and software.

Specific details were given in the description to provide a thorough understanding of the examples. However, it will be understood by one of ordinary skill in the art that the examples may be practiced without these specific details. Accordingly, for example, “based on” is meant to mean “based at least on.”

Also, it is noted that the examples were described as a process, which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional processes or operations not included in the figure. A process may correspond to a method, a function, a procedure, a subroutine, a subprogram, etc. When a process corresponds to a function, its termination corresponds to a return of the function to the calling function or the main function.

Furthermore, examples may be implemented by hardware, software, firmware, middleware, microcode, hardware description languages, or any combination thereof. When implemented in software, firmware, middleware or microcode, the program code or code segments to perform the necessary tasks may be stored in a machine-readable medium, such as a storage medium. A processor(s) may perform the necessary tasks. A code segment may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

While illustrative examples of the disclosure have been described in detail herein, it is to be understood that the inventive concepts may be otherwise variously embodied and employed, and any appended claims are intended to be construed to include such variations, except as limited by the prior art.

What is claimed is:

1. A method comprising:
receiving, at a processor, a request for a health record from a requestor;
obtaining, via the processor, contextual information associated with the request;
based on the contextual information, determining, via the processor, a source of health record information associated with the health record; retrieving, via the processor, the health record information from the source; and
assembling, via the processor, the health record including the retrieved health record information.

2. The method of claim 1, wherein determining the source of the health record information comprises:
parsing text from the contextual information associated with the request;
pattern matching the parsed text from the contextual information to a database of stored health record providers to determine a health record provider that is storing the health record information; and
determining an electronic address of a computing system of the health record provider that stores the health record information.

3. The method of claim 2, further comprising determining, via the processor, eligibility of the requestor to access the health record information, wherein determining eligibility of the requestor comprises cross-referencing, via the processor within a database of requestors, the requestor in order to determine whether the requestor has permission to access information from the source.

4. The method of claim 2, wherein retrieving the health record information from the source comprises:
determining network credentials for the computing system of the health record provider;
establishing a connection via a network to the computing system of the health record provider using the electronic address and the network credentials; and
requesting the health record information from the computing system of the health record provider.

5. The method of claim 1, further comprising:
analyzing the text of the health record information for an indication of additional health record information, wherein the indication of additional health record information comprises an explicit reference or an implicit reference to a document that includes the additional health record information;
obtaining the additional health record information from the document; and
including the additional health record information in the assembled health record.

6. A method comprising:
accessing, at a processor, health record information regarding a patient;
analyzing, by a text analyzer of the processor, the health record information to determine a first reference to additional health record information for the patient;
determining, via the processor, a source of the additional health record information;
retrieving, by the processor via a network, the additional health record information from the source; and
assembling, via the processor, a health record based on the health record information and the retrieved additional health record information.

7. The method of claim 6, wherein analyzing the health record information comprises parsing text of the health record information and using pattern matching rules to identify the first reference to the additional health record information based on the parsed text and keywords stored within a database.

8. The method of claim 6, wherein determining the source of the additional health record information comprises:
using a natural language processing algorithm to identify a health record provider indicated in the analyzed text; and
determining an electronic address for the health record provider, wherein the electronic address indicates a node of a computing system of the source on a network that is storing the additional health record information.

9. The method of claim 6, further comprising:
analyzing, by the text analyzer of the processor, the retrieved additional health record information to determine a second reference to additional supplemental health record information;
determining, via the processor, a second source of the additional supplemental health record information based on the analyzed text associated with the retrieved additional health record information; and
retrieving, by the processor via the network, the additional supplemental health record information from the second source.

10. The method of claim 9, further comprising including the health record information, the retrieved additional health record information, and the retrieved additional supplemental medical information in the assembled health record.

11. The method of claim 10, further comprising redacting portions of the health record based on rules stored in a database regarding the patient associated with the health record.

12. The method of claim 11, wherein the additional health record information comprises lab results and the source of the additional health record information comprises an indication of an electronic address of the source on the network of a computing system associated with a lab that is maintaining the lab results.

13. The method of claim 6, wherein the first reference includes an explicit reference to a document including the additional health record information, and wherein the explicit reference indicates the source.

14. The method of claim 6, wherein the first reference comprises an implicit reference to a document that includes the additional health record information, and wherein contextual information contained within the text of the health record information indicates a health record provider associated with the document.

15. The method of claim 14, wherein determining the source comprises cross-referencing the health record provider with a plurality of health record provider profiles within a database.

16. A health document expansion and retrieval system comprising:
a communication module configured to connect to one or more sources of health records via a network;
a database configured to store one or more health records;
a processor; and
a non-transitory computer readable medium having instructions stored thereon that, when executed by the processor, cause the processor to:
analyze text of health record information using a text analyzer to determine a reference to additional health record information;
determine a source of the additional health record information based on contextual information associated with the health record information; and
retrieve the additional health record information from the source via the communication module.

17. The system of claim 16, wherein, to analyze text of the health record information, the non-transitory computer readable medium includes further instructions that, when executed by the processor, cause the processor to parse text of the health record information and use pattern matching rules to determine the reference to the additional health record information.

18. The system of claim 16, wherein, to analyze text of the health record information, the non-transitory computer readable medium includes further instructions that, when executed by the processor, cause the processor to determine an electronic address of the source based on contextual information associated with the health record information.

19. The system of claim 16, wherein the non-transitory computer readable medium includes further instructions that, when executed by the processor, cause the processor to assemble an electronic health record based on the health record information and the additional health record information.

20. The system of claim 19, wherein the non-transitory computer readable medium includes further instructions that, when executed by the processor, cause the processor to store the electronic health record within the database.
